SEQUENCE LISTING (31 sequences)

SEQ ID NO: 1 - DNA, 800 bp, synthetic construct
  source 1..800: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 2 - DNA, 1728 bp, synthetic construct
  source 1..1728: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 3 - DNA, 399 bp, synthetic construct
  source 1..399: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaaggg

SEQ ID NO: 4 - DNA, 401 bp, synthetic construct
  source 1..401: mol_type = other DNA
tatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 5 - DNA, 799 bp, synthetic construct
  source 1..799: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtataattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 6 - DNA, 799 bp, synthetic construct
  source 1..799: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 7 - DNA, 800 bp, synthetic construct
  source 1..800: mol_type = other DNA
taaacccctccgttttagagaggggttatgctagtcatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 8 - DNA, 799 bp, synthetic construct
  source 1..799: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatatttttcctccttatacttaagccctatagtgagtcgtattaattcgc

SEQ ID NO: 9 - DNA, 800 bp, synthetic construct
  source 1..800: mol_type = other DNA
taaacccctccgttttagagaggggttatgctagttatttgtagagctcatccatgccatgtgtaatcccagcagcagttacaaactcaagaaggtccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 10 - DNA, 799 bp, synthetic construct; identical to SEQ ID NO: 8
  source 1..799: mol_type = other DNA

SEQ ID NO: 11 - DNA, 790 bp, synthetic construct
  source 1..790: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattcgc

SEQ ID NO: 12 - DNA, 799 bp, synthetic construct
  source 1..799: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacactccagtgaaaagttcttctcctttactcatattttttcctccttatacttaagccctatagtgagtcgtattaattcgc

SEQ ID NO: 13 - DNA, 798 bp, synthetic construct
  source 1..798: mol_type = other DNA
taaacccctccgttttagagaggggttatgctagttatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactcatattttttcctccttatacttaagccctatagtgagtcgtattaattc

SEQ ID NO: 14 - DNA, 1725 bp, synthetic construct
  source 1..1725: mol_type = other DNA
atgaagcatatgccgagaaagatgtatagttgtgactttgagacaactactaaagtggaagactgtagggtatgggcgtatggttatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagtacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 15 - DNA, 1729 bp, synthetic construct
  source 1..1729: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaagggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 16 - DNA, 1727 bp, synthetic construct
  source 1..1727: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagagatagctaaagactttaaactaactgttcttaaaggtgatattgattaccacaaagaaagaccagtcggctataagataacacccgaagaatacgcctatattaaaaacgatattcagattgttgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 17 - DNA, 1727 bp, synthetic construct
  source 1..1727: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcagggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 18 - DNA, 1728 bp, synthetic construct
  source 1..1728: mol_type = other DNA
atgaagcatatgccgagaaagatgtatagttgtgactttgagacaactactaaagcggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 19 - DNA, 1728 bp, synthetic construct
  source 1..1728: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagactacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 20 - DNA, 1728 bp, synthetic construct
  source 1..1728: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagcttatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 21 - DNA, 1726 bp, synthetic construct
  source 1..1726: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagaggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 22 - DNA, 1729 bp, synthetic construct
  source 1..1729: mol_type = other DNA
atgaagcatatgccgagaaagatgtatagttgtgactttgagacaactactaaagtggaagactgtagggtatgggcgtatggttatatgaatatagaagatcacagtgagtacaaaataggtaatagcctggatgagtttatggcgtgggtgttgaaggtacaagctgatctatatttccataacctcaaatttgacggagcttttatcattaactggttggaacgtaatggttttaagtggtcggctgacggattgccaaacacatataatacgatcatatctcgcatgggacaatggtacatgattgatatatgtttaggctacaaagggaaacgtaagatacatacagtgatatatgacagcttaaagaaactaccgtttcctgttaagaagatagctaaagactttaaactaactgttcttaaaggtgatattgattaccacaaagaaagaccagtcggctataagataacacccgaagaatacgcctatattaaaaacgatattcagattattgcggaacgtctgttaattcagtttaagcaaggtttagaccggatgacagcaggcagtgacagtctaaaaggtttcaaggatattataaccactaagaaattcaaaaaggtgtttcctacattgagtcttggactcgataaggaagtgagatacgcctatagagggtggttttacatggttaaatgataggttcaaagaaaaagaaatcggagaaggcatggtcttcgatgttaatagtctatatcctgcacagatgtatagccgtctccttccatatggtgaacctatagtattcgagggtaaatacgtttgggacgaagattacccactacacatacagcatatcagatgtgagttcgaattgaaagagggctatatacccactatacagataaaaagaagtaggttttataaaggtaatgagtacctaaaaagtagcggcggggagatagccgacctctggttgtcaaatgtagacctagaattaatgaaagaacactacgatttatataacgttgaatatatcagcggcttaaaatttaaagtaactacaggtttgtttaaagattttatagataaatggacgtacatcaagacgacatcagaaggagcgatcaagcaactagcaaaactgatgttaaacagtctatacggtaaattcgctagtaaccctgatgttacagggaaagtcccttatttaaaagagaatggggcgctaggtttcagacttggagaagaggaaacaaaagaccctgtttatacacctatgggcgttttcatcactgcatgggctagatacacgacaattacagcggcacaggcttgttatgatcggataatatactgtgatactgacagcatacatttaacgggtacagagatacctgatgtaataaaagatatagttgaccctaagaaattgggatactgggcacatgaaagtacattcaaaagagctaaatatctgagacagaagacctatatacaagacatctatatgaaagaagtagatggtaagttagtagaaggtagtccagatgattacactgatataaaatttagtgttaaatgtgcgggaatgactgacaagattaagaaagaggttacgtttgagaatttcaaagtcggattcagtcggaaaatgaagcctaagcctgtgcaagtgccgggcggggtggttctggttgatgacacattcacaatcaaataa

SEQ ID NO: 23 - DNA, 20 bp, synthetic construct
  source 1..20: mol_type = other DNA
gcgaattaatacgactcact

SEQ ID NO: 24 - DNA, 20 bp, synthetic construct
  source 1..20: mol_type = other DNA
aaacccctccgttttagaga

SEQ ID NO: 25 - DNA, 16 bp, synthetic construct
  source 1..16: mol_type = other DNA
gtaaaacgacggccag

SEQ ID NO: 26 - DNA, 17 bp, synthetic construct
  source 1..17: mol_type = other DNA
caggaaacagctatgac

SEQ ID NO: 27 - DNA, 20 bp, synthetic construct
  source 1..20: mol_type = other DNA
atgaagcatatgccgagaaa

SEQ ID NO: 28 - DNA, 25 bp, synthetic construct
  source 1..25: mol_type = other DNA
ttatttgattgtgaatgtgtcatca

SEQ ID NO: 29 - DNA, 19 bp, synthetic construct
  source 1..19: mol_type = other DNA
atgacagcaggcagtgaca

SEQ ID NO: 30 - DNA, 20 bp, synthetic construct
  source 1..20: mol_type = other DNA
gctagttgcttgatcgctcc

SEQ ID NO: 31 - DNA, 100 bp, synthetic construct
  source 1..100: mol_type = other DNA
gttgtttgcccctcccccgtgccttccttgaccctggaaggtgccactcccactgtcctttcctaataaaatgaggaaattgcatcacaacactcaacct

(19) United States
(12) Patent Application Publication
(10) Pub. No.: US 2026/0015380 A1
(43) Pub. Date: Jan. 15, 2026

(54) LONG OLIGO SYNTHESIS

(71) Applicant: Shiyue Fang, Houghton, MI (US)

(72) Inventor: Shiyue Fang, Houghton, MI (US)

(21) Appl. No.: 19/260,418

(22) Filed: Jul. 4, 2025

Related U.S. Application Data
(60) Provisional application No. 63/670,615, filed on Jul. 12, 2024.

(51) Int. Cl. C07H 21/00; C07H 1/00; C12P 19/34

(52) U.S. Cl. CPC C07H 21/00; C07H 1/00; C12P 19/34

(57) ABSTRACT

Solid supports and methods using the solid supports for de novo long oligo synthesis are disclosed. Long oligos with more than 100 nucleotides are notably difficult to synthesize. The challenges include low percentage yield, high error rate, and difficulty to isolate the correct sequence. Using the disclosed solid supports and their associated methods, oligos with more than 1,000 nucleotides can be readily obtained. The synthesis of the 1,728-nucleotide Φ29 DNA polymerase gene was demonstrated.

### CROSS-REFERENCE TO RELATED APPLICATIONS

This application for patent claims priority to Provisional Application Ser. No. 63/670,615 filed on Jul. 12, 2024.

### FEDERALLY SPONSORED RESEARCH

This invention was made with United States government support under grant numbers 1954041 and GM109288 awarded by the National Science Foundation and National Institutes of Health, respectively. The government has certain rights in the invention.

### REFERENCE TO ELECTRONICALLY FILED SEQUENCE LISTING

The content of the electronically submitted Nucleotide Sequence Listing XML file (Name: OligoSequences.xml; Size: 53 KB; Date of Creation: Jun. 30, 2025) is herein incorporated by reference in its entirety.

### BACKGROUND OF THE INVENTION

The present invention pertains to the field of de novo oligonucleotide (commonly called oligo) synthesis. Oligos including oligodeoxyribonucleotides (ODNs), oligoribonucleotides (ORNs), modified oligos, and their hybrids have applications in many fields. In some of the fields such as synthetic biology [Jones et al 2024 Nat Rev Microbiol 22:345 doi:10.1038/s41579-023-01007-9],nucleic acid vaccines [Xu et al 2024 Vaccines 12:664 doi:10.3390/vaccines12060664],oligonucleotide therapeutics [Obexer et al 2024 Science 384: eadl4015doi:10.1126/science.adl4015], gene editing [Villiger et al 2024 Nat Rev Mol Cell Bio 25:160 doi:10.1038/s41580-023-00697-6], protein engineering [Wang et al 2024 Chem Eur J 30:e202303889 doi:10.1002/chem.202303889], and digital data storage [Ceze et al 2019 Nat Rev Genet 20:456 doi:10.1038/s41576-019-0125-3], oligos longer than 100 nucleotides (abbreviated as nt, 100-mer) including those with multiple thousands or millions of nucleotides are needed. The state-of-the-art of de novo chemical oligo synthesis technologies, primarily using the phosphoramidite chemistry, can only produce oligos with length up to ˜200-mer for ODN and ˜120-mer for ORN [Hoose et al 2023 Nat Rev Chem 7:144 doi:10.1038/s41570-022-00456-9]. In addition, when the length of the oligos reaches the 200-mer and 120-mer limits, the quality, characterized by overall yields and error rates, is low.

Currently, ODNs longer than 200-mer, and in many cases, even shorter than 200-mer, are produced by linking short ODNs, usually 20-mer to 60-mer, together using biological means such as PCR assembly and ligation [Miklos et al 2012 Curr Protoc Mol Biol doi:10.1002/0471142727.mb0323s99]. The short ODNs are produced by de novo chemical synthesis, primarily using the phosphoramidite chemistry. The drawbacks of the biological means have been widely recognized [Miklos et al 2012 Curr Protoc Mol Biol doi:10.1002/0471142727.mb0323s99; Eroshenko et al 2012 Curr Protoc Chem Biol doi:10.1002/9780470559277.ch110190]. In addition to high cost, multiple-step procedures, long turn-around time, proneness to errors, and difficulty for automation and parallelization, it is challenging to use them to produce sequences with difficult elements. Such difficult elements include stable higher order structures, long repeats, high or low G/C contents, and site-specific modifications. Stable higher order structures include but are not limited to hairpin, cruciform, and G-quadruplex. They widely exist in DNAs and are required for biological functions [Kolmogorov et al 2019 Nat Biotechnol 37:540 doi:10.1038/s41587-019-0072-8]. They are difficult elements in terms of PCR assembly because they may affect the activity of enzymes. For long repeats, if the repeats are longer than the short ODNs used for assembling the long ODNs, the assembly would not be accurate. For high or low G/C contents, they can cause promiscuous annealing and secondary structures of ODNs making long oligo assembly error-prone. For site-specific modifications, even though they can be incorporated into the short oligos, during PCR assembly, all will be erased.

Currently, ORNs longer than 120-mer, and in many cases, even shorter than 120-mer, are primarily produced by in vitro transcription from ODNs [Taemaitree et al 2019 Nat Commun 10:1610 doi:10.1038/s41467-019-09600-4]. Besides drawbacks such as complex procedure and high cost, similar to long ODN assembly using biological means, it is challenging to produce sequences with difficult elements such as stable higher order structure, long repeats, high or low G/C contents, and site-specific modifications. Long ORNs may also be produced by ligation of chemically synthesized or in vitro transcribed short ORNs such as 20-mers and 50-mers. However, ORN ligation is more complex than ODN ligation. Its efficiency can be lower, and ligation side products may be generated [Stark et al 2014 Methods Mol Biol 1126:137 doi:10.1007/978-1-62703-980-2_10].

There are several reasons for having not used the phosphoramidite chemistry to produce longer oligos directly by the community. Common wisdom believed that for de novo stepwise chemical long oligo synthesis, the overall yield is a problem. For example, if the average stepwise yield is 99.5%, for a 200-mer synthesis, the overall yield is only 37%; for a 500-mer synthesis, it drops to 8%; and for a 1,000-mer synthesis, it is 0.7%. The low overall yields not only result in low quantities of target oligos, but also imposes insurmountable challenges for product isolation and purification. Beyond the common beliefs related oligo length, yield and purification, for direct chemical long oligo synthesis, high error rate is an even more serious problem [Masaki et al 2022 Sci Rep 12:12095 doi:10.1038/s41598-022-16222-2]. This problem is even more true and more challenging to address. The errors include substitution errors such as G-to-A, G-to-T, C-to-T, T-to-C, A-to-G, and A-to-T, single nt deletion, block nt deletion, and single nt insertion. The total error rate can be as high as 0.58% for ODN synthesis, which makes the selection of a completely correct oligo molecule challenging even for a sequence as short as 200-mer [Masaki et al 2022 Sci Rep 12:12095 doi:10.1038/s41598-022-16222-2].

To address the problem of the lack of ideal technologies for the production of long oligos, significant resources from academia and industry have been devoted to the development of the template-independent enzymatic oligo synthesis (TiEOS) technologies [Hoose et al 2023 Nat Rev Chem 7:144 doi:10.1038/s41570-022-00456-9; Lee et al 2019 Nat Commun 10:2383 doi:10.1038/s41467-019-10258-1; Eisenstein 2020 Nat Biotechnol38:1113 doi:10.1038/s41587-020-0695-9]. These technologies primarily use carefully engineered terminal deoxynucleotidyl transferases (TdT) to link nucleotide monomers stepwise to the oligo chain anchored to a solid support. The efforts further demonstrate the need for better technologies for de novo long oligo synthesis. While the TiEOS technologies hold great promise, they still have various drawbacks. For example, the molecular weight of TdT is ˜58,000 while that of nucleotide to be incorporated is only ˜500. In almost all known TiEOS methods, stoichiometric or large excess enzymes are used in each coupling step. Therefore, the methods are not atom economic. In addition, the enzymatic methods may have the same problems as PCR assembly and ligation, and may not be able to synthesize oligos with stable higher order structures and site-specific modifications.

In summary, currently, chemical methods cannot produce ODNs longer than 200-mer and ORNs longer than 120-mer. Long oligos are produced with biological means such as PCR assembly, ligation and in vitro transcription. Besides drawbacks such as high cost, long turn-around time and others, the methods cannot produce long oligos with difficult elements. To address the problem, large resources have been devoted to the development of the TIEOS methods, but these methods can still not solve all the problems. Therefore, there is a need for more innovation in the field of de novo long oligo synthesis.

### BRIEF SUMMARY OF THE INVENTION

This invention is related to solid supports comprising smooth surface for solid phase oligo synthesis. The new supports are intended to address the long-lasting problem of de novo long oligo synthesis. In contrast to traditional solid supports such as controlled pore glass (CPG) and highly cross-linked polystyrene [Mccollum et al 1991 Tetrahedron Lett 32:4069 doi:10.1016/S0040-4039(00)79865-0], using which oligos synthesis occurs almost entirely within pores, the solid supports in this invention have no intentionally installed pores, and oligo synthesis is conducted on smooth surface of the supports. The difference of the results between conducting oligo synthesis on smooth surface and within pores are drastic. Using traditional supports, only short oligos—up to ˜200-mer for ODN, and up to ˜120-mer for ORN—can be synthesized [Yin et al 2025 Chem Sci 16:1966 doi:10.1039/d4sc06958g] . Using the new supports, the synthesis of a 800-mer (SEQ ID NO: 001) and a 1,728-mer (SEQ ID NO: 002) has been successfully demonstrated. It is predicted that oligos even longer than 1,728-mer can be synthesized as well. Surprisingly, using the new supports, not only longer oligos can be synthesized, but also the error rates, such as the substitution, single nucleotide deletion, block deletion, and single nucleotide addition error rates, are all drastically reduced [Yin et al 2025 Chem Sci 16:1966 doi:10.1039/d4sc06958g].

The solid supports comprising smooth surface in this invention include but are not limited to non-porous glass wool, glass beads, highly cross-linked polymers [Mccollum et al 1991 Tetrahedron Lett 32:4069 doi:10.1016/S0040-4039(00)79865-0; Murata et al 2006 Tetrahedron Lett 47:2147 doi:10.1016/j.tetlet.2006.01.135], and carbon fiber. The supports may also be the inner wall of an oligo synthesis column, tube or capillary. In some embodiments, the supports are functionalized to have a cleavable linker or linkers attached to the surface. Through the linker or linkers, oligos are synthesized stepwise. After synthesis, the oligo product is obtained by cleaving the linker or linkers. In some embodiments, at the end of the synthesis, a polymerizable group or groups are attached to the full-length sequence but not to the failure sequences, and the full-length sequence is purified or isolated from the complex crude mixture using a process called catching-by-polymerization (CBP) [Fang 2010 Patent:U.S. Pat. No. 7,850,949; Pokharel et al 2016 Green Chem 18:1125 doi:10.1039/c5gc01762a; Eriyagama et al 2018 Org Process Res Dev 22:1282 doi:10.1021/acs.oprd.8b00209]. In some embodiments, CBP is not conducted. Instead, no purification or purification using other methods is carried out. In some embodiments, oligo synthesis involves phosphoramidite chemistry. In some embodiments, oligo synthesis involves enzymatic method or methods [Hoose et al 2023 Nat Rev Chem 7:144 doi:10.1038/s41570-022-00456-9; Kolmogorov et al 2019 Nat Biotechnol 37:540 doi:10.1038/s41587-019-0072-8; Lee et al 2019 Nat Commun 10:2383 doi:10.1038/s41467-019-10258-1; Eisenstein 2020 Nat Biotechnol38:1113 doi:10.1038/s41587-020-0695-9; Barthel et al 2020 Genes 11:102 doi:10.3390/genes 11010102; Perkel 2019 Nature 566:565 doi:10.1038/d41586-019-00682-0; Gibson et al 2009 Nat Methods 6:343 doi:10.1038/nmeth. 1318]. In some embodiments, both chemical and enzymatic methods could be used.

### ADVANTAGES

Compared with traditional support with intentionally installed pores primarily for the purpose of increasing oligo loading, the new solid supports comprising smooth surface have the advantage of being capable of synthesizing much longer oligos.

Compared with traditional support with intentionally installed pores primarily for the purpose of increasing oligo loading, the new solid supports comprising smooth surface have the advantage of being capable of synthesizing oligos with drastically reduced error rates [Yin et al 2025 Chem Sci 16:1966 doi:10.1039/d4sc06958g].

Compared with oligo synthesis on flat surface such as in the cases for producing microarrays on a chip, the new solid support can be used to produce larger quantities of oligos. The quality of the oligos is also better because of better reagent control. In addition, long oligo synthesis on flat surface has not been demonstrated.

Compared with oligo synthesis on the surface of wells of multiple well plates, the new supports, which are packed in synthesis columns, arranged within a column, or are the inner wall surfaces of synthesis columns, tubes and capillaries, can produce higher quality and longer oligos due to better reagent control. In addition, long oligo synthesis in wells has not been demonstrated.

Compared with oligo synthesis on soluble polymers [Bonora 1995 Appl Biochem Biotech 54:3; Kim et al 2013 Chem Eur J 19:8615 doi:10.1002/chem.201300655], using the new supports comprising smooth surface, oligo synthesis is easier to automate because the former needs precipitation or special means such as nano-filtration to separate the oligos from side products and excess reagents. In addition, long oligo synthesis on soluble polymers has not been demonstrated.

Compared with oligo synthesis on organic molecules [Rosenqvist et al 2024 J Org Chem 89:13005 doi:10.1021/acs.joc.4c01053], using the new supports comprising smooth surface, oligo synthesis is easier to automate because the former needs precipitation or special means such as nano-filtration to separate the oligos from side products and excess reagents. In addition, long oligo synthesis on organic molecules has not been demonstrated.

Compared with oligo synthesis in the matrix of swellable polymers such as the cross-linked polystyrene based Primer Support™ 5G DNA/RNA synthesis support produced by Cytiva (Marlborough, MA) [Catani et al 2020 Biotechnol J 15:e1900226 doi:10.1002/biot.201900226; Menéndez-Méndez et al 2024 Results Chem 12:101930 doi:10.1016/j.rechem.2024.101930], using the new supports comprising smooth surface, oligo synthesis is more efficient because the former gives slower reaction kinetics and the latter gives higher reaction kinetics. In addition, long oligo synthesis in the matrix of swellable polymers is expected to have problems of steric hindrance and high error rates. Further, long oligo synthesis in the matrix of swellable polymers has not been demonstrated.

Compared with oligo synthesis on the surface of nanoparticles, using the new supports comprising smooth surface, oligo synthesis is easier to automate because nanoparticles packed in a synthesis column would not be easy for reagents and solvents to pass through the column, and thus more complex means would be needed for oligo synthesis automation. In addition, long oligo synthesis on nanoparticles has not been demonstrated.

Compared with oligo synthesis using core-shell solid supports [Menéndez-Méndez et al 2024 Results Chem 12:101930 doi:10.1016/j.rechem.2024.101930], using the new supports comprising smooth surface, the kinetics of the reactions may be faster, and thus more suitable for long oligo synthesis. In addition, long oligo synthesis on core-shell solid supports has not been demonstrated.

Compared with oligo synthesis on the surface of colloids with magnetic cores [Benner 1987 Patent:U.S. Pat. No. 4,638,032], using the new supports comprising smooth surface, oligo synthesis is easier to automate because using colloids with magnetic cores would need more complex means for the separation of support carrying the oligo product from side products, excess reagents and solvents. In addition, long oligo synthesis on colloids with magnetic pores has not been demonstrated.

### DEFINITIONS

Carbon based materials, in this application, include carbon fiber, carbon particles and other forms of materials primarily comprised of carbon. These materials may be used in packed beds or as monolithic structures, and are suitable for reagent flow under pressure or gravity in oligonucleotide synthesis.

Enzymatic methods, in this application, refers to de novo oligo synthesis by connecting nucleotides or oligonucleotides stepwise using enzymatic reactions. Such enzymatic methods have been used for ODN and ORN synthesis [Hoose et al 2023 Nat Rev Chem 7:144 doi:10.1038/s41570-022-00456-9; Lee et al 2019 Nat Commun 10:2383 doi:10.1038/s41467-019-10258-1; Eisenstein 2020 Nat Biotechno/38:1113 doi:10.1038/s41587-020-0695-9; Barthel et al 2020 Genes 11:102 doi:10.3390/genes11010102; Gibson et al 2009 Nat Methods 6:343 doi:10. 1038/Nmeth. 1318; Law et al 2024 ACS Catal 14:12318 doi:10.1021/acscatal.4c00756; Wiegand et al 2025 Nat Biotechnol 43:762 doi:10.1038/s41587-024-02244-w; Jensen et al 2018 Biochem 57:1821 doi:10.1021/acs.biochem.7b00937; Jarchow-Choy et al 2011 Nucleic Acids Res 39:1586 doi:10.1093/nar/gkq853; Kobayashi et al 2016 Chem Commun 52:3762 doi:10.1039/c5cc10039a; Motea et al 2010 BBA Proteins Proteom 1804:1151 doi:10.1016/j.bbapap.2009.06.030; Pichon et al 2024 Chem Eur J 30:e202400137 doi:10.1002/chem.202400137; Gao et al 2025 Biotech Adv 82:108604 doi:10.1016/j.biotechadv.2025.108604; Sabat et al 2023 Front Chem 11:1161462 doi:10.3389/fchem.2023.1161462; Forget et al 2025 Nucleic Acids Res 53:gkaf115 doi:10.1093/nar/gkaf115; Wang et al 2022 ACS Synth Biol 11:4142 doi:10.1021/acssynbio.2c00456; Mitton-Fry et al 2024 Chem Sci 15:13068 doi:10.1039/d4sc03769c]. The enzymatic reactions may also be ligation of oligonucleotides with or without a template strand.

Flat surface, defined in this application, is a surface without intentionally made curvatures or pores, and extends in two dimensions beyond ˜100 μm. In particular, it refers to the surface of DNA or RNA microarrays.

Highly cross-linked polymer, defined in this application, is a type of polymer in which the individual polymer chains are extensively interconnected through covalent bonds, forming a dense, three-dimensional network. The material is insoluble or minimally swellable in the solvents used for oligo synthesis. For example, highly cross-linked polystyrene is widely used for oligo synthesis.

Loading, in this application, refers to the mole quantity of attachment sites on the surface of a specific quantity of a solid support that can be used for oligo synthesis. Its unit can be mmol/g, μmol/g or nmol/g, etc. The surface can be a smooth surface or the surface within pores.

Nanoparticle, defined in this application, is a particle with a diameter smaller than 1 μm.

Oligo is an abbreviation of oligonucleotide, which include oligodeoxyribonucleotide (ODN), and oligoribonucleotide (ORN). It may also include modified ODN and ORN, and combination of them.

Oligonucleotide is a chain of nucleotides connected by phosphate diester bonds or other covalent linkages. Other forms of inter-nucleotide linkages are also possible. The nucleotides may be modified.

Pores, in this application, refers those that are intentionally included in the solid support for the purpose of increasing surface area and thus loading for oligo synthesis. Their dimension may be larger than 200 Å in every direction.

Smooth surface, in this application, refers to the surface of solid supports, including those with various shapes (e.g., sphere, fiber, rod, disc, square plate, cube), that lacks intentionally introduced pores or microstructures designed to increase surface area. It may contain minor surface irregularities due to manufacturing processes.

Solid support, in this application, refers to materials that are insoluble under oligonucleotide synthesis conditions and on which oligonucleotide synthesis occurs with the oligonucleotide covalently or physically attached during synthesis. The support may be used in packed beds, columns, flow cells, or other formats.

Steric hindrance, in this application, refers to the restriction of reagents and solvents to reach the reaction sites in the pores of solid support for oligo synthesis due to limited space in the pores. It also refers to the restriction of the movement of the oligo molecules in the pore due to limited space. The result of steric hindrance is that the reactions for oligo synthesis may be slower, or do not occur, and in the washing steps, the washing is less effective.

Substantially smooth surface, in this application, refers to surfaces that do not contain intentionally introduced pores or surface features designed to increase internal surface area for the purpose of increasing loading. It does not mean that it is perfectly smooth, and may have manufacturing-related imperfections.

### BRIEF DESCRIPTION OF DRAWINGS

FIG. 1. Catching-by-polymerization (CBP) oligo purification.

FIG. 2. Workflow for the characterization of long oligos.

FIG. 3. Functionalization of glass wool and glass beads.

FIG. 4. Functionalization of carbon fiber.

FIG. 5. Determination of the loading of carbon fiber.

FIG. 6. Images showing trityl color of last detritylation before coupling with PTP.

FIG. 7. Gel electrophoresis images of PCR products using CBP-purified oligos as templates.

FIG. 8. Gel electrophoresis images of colony PCR products.

FIG. 9. Synthesis of linker phosphoramidite.

FIG. 10. The trityl color on glass wool (left) and glass beads (right) during the detritylation steps for the conversion of [F008] to [F001].

### DETAILED DESCRIPTION OF THE INVENTION

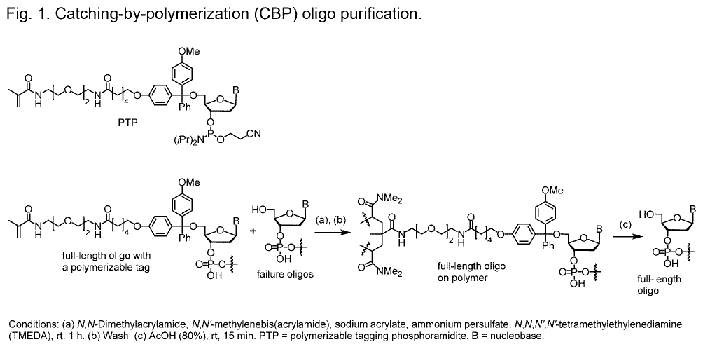

Since 2010, we have been developing a new method called catching-by-polymerization (CBP) for the purification of oligos (FIG. 1) as well as peptides [Zhang et al 2014 Org Lett 16:1290 doi:10.1021/ol403426u; Fang et al 2010 Org Lett 12:3720 doi:10.1021/ol101316g]. The method works by attaching a polymerizable group such as the methacrylamide group using a polymerizable tagging phosphoramidite (PTP) to the full-length ODNs at the end of automated solid phase synthesis. Because failure sequences are capped with reagents such as acetic anhydride in each synthetic cycle, the attachment does not occur on them. After ODN deprotection and cleavage, the crude mixture mainly contain the full-length ODN, which has a polymerizable group, and the failure sequences, which do not contain a polymerizable group (FIG. 1). To separate the two, the crude is mixed with a few drops of polymerization solution containing N,N-dimethylacrylamide, and N,N′-methylenebis(acrylamide). The radical acrylamide polymerization reaction is then initiated. The full-length ODN is co-polymerized into the polyacrylamide gel, while the failure sequences as well as other impurities remain in solution. Purification is then achieved by washing the gel with water to remove the failure sequences and other impurities, followed by cleaving the full-length ODN from the polymer (FIG. 1) [Pokharel et al 2016 Green Chem 18:1125 doi:10.1039/c5gc01762a].

Over the years, the method has evolved to the level capable of isolating chemically synthesize ODNs from crude mixtures with unlimited complexity [Fang 2010 Patent: U.S. Pat. No. 7,850,949; Yuan et al 2012 RSC Adv 2:2803 doi:10. 1039/c2ra01357f]. A few years ago, we realized that the method might be able to address the challenge of isolating the full-length ODN product, of which the yield might be extremely low, from the complex crude mixture of direct ultralong chemical ODN synthesis [Pokharel et al 2016 Green Chem 18:1125 doi:10.1039/c5gc01762a; Eriyagama et al 2018 Org Process Res Dev 22:1282 doi:10.1021/acs.oprd.8b00209]. Contrary to the prevailing wisdom that direct chemical synthesis of ultralong ODN is infeasible due to its perceived extremely low overall yield, we believed that with CBP, we could isolate the target ODN, and the low percentage yield was not a problem for many biological applications. For example, for a 1,000-mer ODN synthesis, with 99.5% average stepwise yield, the overall yield would be 0.7%. For a 1 μmol synthesis, the quantity of the target ODN would be 7 nmol, which would be 7,000 times the 1 μmol quantity needed for many biological applications. With these considerations, we went ahead to synthesize ultralong ODNs using the traditional solid support controlled pore glass (CPG) [Yin et al 2025 Chem Sci 16:1966 doi:10.1039/d4sc06958g; Yin et al 2023 Beilstein J Org Chem 19:1957 doi:10.3762/bjoc. 19.146].

Using CPG [Rothstein et al 2012 Genet Eng Biotechn News 32:42 doi:10.1089/gen.32.9.20] as well as other known solid supports such as porous highly cross-linked polystyrene [Mccollum et al 1991 Tetrahedron Lett 32:4069 doi:10.1016/S0040-4039(00)79865-0], ODN synthesis is conducted in the pores. Common wisdom believes that conducting synthesis in pores is needed. Otherwise, the quantity of product would be too low for practical applications. However, for long ODN synthesis, conducting synthesis in pores would have the problem of steric hindrance due to the large sizes of ODN molecules. To address this problem, we reduced the loading of commercial CPG to 50% so that each pore had less number of ODN molecules, and the steric hindrance could be reduced [Yin et al 2023 Beilstein J Org Chem 19:1957 doi:10.3762/bjoc. 19.146]. With this adjustment, we synthesized a 399-mer (SEQ ID NO: 003) and 401-mer (SEQ ID NO: 004). The overall yields were indeed extremely low (˜2%). However, as we predicted, using CBP, we indeed were able to isolate the full-length ODN products [Yin et al 2023 Beilstein J Org Chem 19:1957 doi:10.3762/bjoc. 19.146].

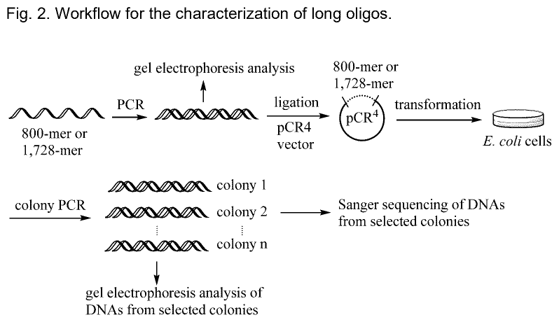

For the characterization of the ultralong ODNs (FIG. 2), the CBP-purified ODNs (SEQ ID NOs: 003 and 004) were amplified with PCR, ligated into a vector, which was transformed into E. coli cells. Many colonies of the cells were grown, and colony PCR was performed. The long ODNs from selected colonies were subjected to Sanger sequencing. Gratifyingly, a few colonies were found to have completely correct sequence of the 399-mer (SEQ ID NO: 003) and 401-mer (SEQ ID NO: 004). However, the ODNs from the majority of the colonies had one or more errors which included substitution, single nt deletion, block nt deletion, and single nt insertion errors. Among them, the block nt deletion, which spans from two to hundreds of nt, was most prevalent [Yin et al 2023 Beilstein J Org Chem 19:1957 doi:10.3762/bjoc. 19.146]. To synthesize even longer ODNs, the number of block nt deletion errors must be drastically reduced. In addition, the loading must be further reduced to give more space in the pores of CPG for ODN growth.

At that point, we had two directions for the research. One was to figure out the cause of the nt block deletion errors, which could be the loss of capping from failure sequences after many synthesis cycles as well as others, and continue to use CPG with further reduced loading as solid support for long oligo synthesis. The other was to avoid using a large amount of resources to figure out the cause of nt block deletion errors, and focus on eliminating steric hindrance caused by conducting synthesis in pores completely by conducting synthesis on smooth surface. The latter direction is against common wisdom because it is widely accepted that conducting synthesis on smooth surface would give too little product. However, as it turned out later, the latter was the right choice, which besides solving the steric hindrance problem, unexpectedly, solved the nt block deletion error problem and drastically reduced other errors as well. We were fortunate for having chosen the right direction. Otherwise, we might not be able to make the discoveries in this invention. Because the need for long ODNs has been known for several decades, had we not made the right choice, the discoveries in this disclosure might have had to be made much later or could not be made in a foreseeable future.

To minimize the risk, before investing resources into the endeavor, we calculated the loading of glass wool, glass beads and carbon fiber, which could potentially be the solid supports with smooth surface for oligo synthesis [Yin et al 2025 Chem Sci 16:1966 doi:10.1039/d4sc06958g]. For glass wool and glass fiber, their geometry is about the same. Their diameters are ˜8 μm. For glass beads, to allow liquid to be able to flow through a synthesis column filled with them with acceptable resistance, a condition required for automated solid phase synthesis, the diameter needs to be ˜30 μm or larger. We chose 58 μm for our calculation. Other parameters and details for the calculation can be found in our published paper [Yin et al 2025 Chem Sci 16:1966 doi:10.1039/d4sc06958g]. The calculated loading for glass wool and carbon fiber was 1.208 μm/g, and that for glass beads was 0.249 μm/g. These values were actually not far away from the loading of the CPG we were using in our lab, which was 5.405 μm/g as given by the manufacturer of the material.

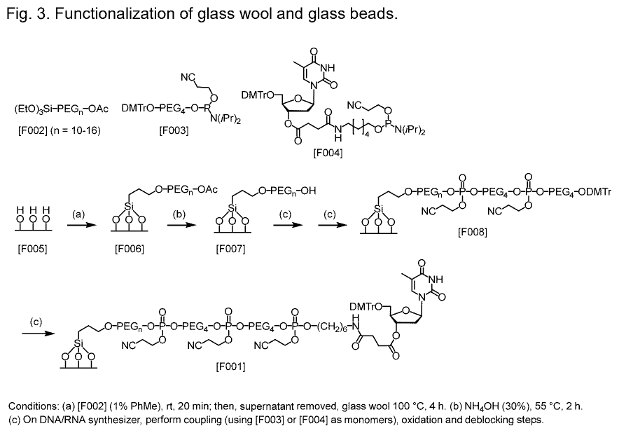

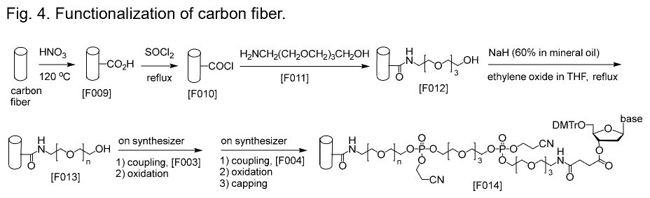

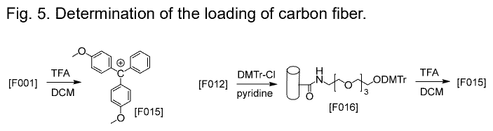

As analyzed earlier, the low overall percentage yield for ultralong ODN synthesis is not a problem as significant as common wisdom suggests because even for a low yield such as 0.7%, for a 1 μm synthesis, 7 nmol ODN can still be obtained. Using glass wool, carbon fiber or glass beads, the loading is lower, but with the quantities of the solid supports similar to the quantities of CPG that is typically used, the quantities of ODN product is still in the hundreds of pmol range, which is sufficient for numerous biological applications [Kuhn et al 2017 Eng Life Sci 17:6 doi:10.1002/elsc.201600121; Kim et al 2011 J Biotechnol151:319 doi:10.1016/j.jbiotec.2011.01.004]. With these considerations, we went ahead to functionalize glass wool, glass beads (FIG. 3) and carbon fiber (FIG. 4). We further measured the loading of the solid supports experimentally using a trityl assay (FIG. 5) [Guzaev et al 2013 Curr Protoc Nucleic Acid Chem doi:10.1002/0471142700.nc0302s52]. The measured values, 0.981 μm/g for glass wool, 0.256 μm/g for glass beads, and 1.79 μm/g for carbon fiber, were close to the calculated values [Yin et al 2025 Chem Sci 16:1966 doi:10.1039/d4sc06958g].

For the functionalization of glass wool and glass beads, the chemistry is the same (FIG. 3). The glass [F005] was first treated with a piranha solution, and then reacted with compounds [F002] to give [F006] [Khanal et al 2016 Chem Eur J 22:9760 doi:10.1002/chem.201600982], which was converted to [F007] by removing the acetyl group. [F007] was converted to [F008] on a DNA synthesizer using [F003] as the coupling reagent. Further coupling on a DNA synthesizer using monomer [F004] gave the desired glass wool or glass beads solid support with a smooth surface [F001]. Loading of the supports was determined by measuring UV absorption of DMTr cation after treating [F001] with an acid (FIG. 5) [Guzaev et al 2013 Curr Protoc Nucleic Acid Chem doi:10.1002/0471142700.nc0302s52].

For the functionalization of carbon fiber, many potential methods could be tested. Examples include those involving nBuLi, free radicals, carbene, nitrene, nitrile imine, ylide, ozonolysis, cycloaddition, and electrophilic addition reactions [Diez-Pascual 2021 Macromol 1:64 doi:10.3390/macromol1020006; Gao et al 2013 Carbon 54:133 doi:10.1016/j.carbon.2012.11.010; Diez-Pascual et al 2012 Carbon 50:857 doi:10.1016/j.carbon.2011.09.046; Wu et al 2010 J Mater Chem 20:1036 doi:10.1039/b911099m; Park et al 2006 Chem Mater 18:1546 doi:10.1021/cm0511421; Hong et al 2005 Chem Mater 17:2247 doi:10.1021/cm0480541; Long et al 2008 Chem Commun 2788 doi:10.1039/b719380g; Yan et al 2015 Chem Soc Rev 44:3295 doi:10. 1039/c4cs00492b; Wu et al 2003 Macromol 36:6286 doi:10.1021/ma034513c; Darabi et al 2012 Appl Surf Sci 258:8953 doi:10.1016/j.apsusc.2012.05.126; Blake et al 2004 J Am Chem Soc 126:10226 doi:10.1021/ja0474805; Peng et al 2003 J Am Chem Soc 125:15174 doi:10.1021/ja037746s]. We chose the method involving nitric acid (FIG. 4) [Zhang et al 2018 Polymers 10:1171 doi:10.3390/polym10101171; Eyckens et al 2021 J Mater Chem A 9:26528 doi:10.1039/d1ta07151c]. Treating carbon fiber with nitric acid at 120° C. gave [F009], which was converted to [F010] using thionyl chloride. Treating [F010] with [F011] gave [F012]. To increase the length of the linker and suppress effects of hydrophobicity and the extended IT system of carbon fiber surface on oligo synthesis, [F012] was converted to with n more than 100 using ethylene oxide in the presence of NaH. On a DNA synthesizer, coupling [F013] with [F003] and [F004] as described for glass wool and glass bead functionalization gave the target carbon fiber [F014] that is suitable for ODN synthesis. For the determination of the loading of carbon fiber, [F012] was treated with DMTr-CI to give [F016]. Loading was them determined by treating [F016] with acid followed by measuring the UV absorption of the DMTr cation [F015] as described in the literature [Guzaev et al 2013 Curr Protoc Nucleic Acid Chem doi:10.1002/0471142700.nc0302s52].

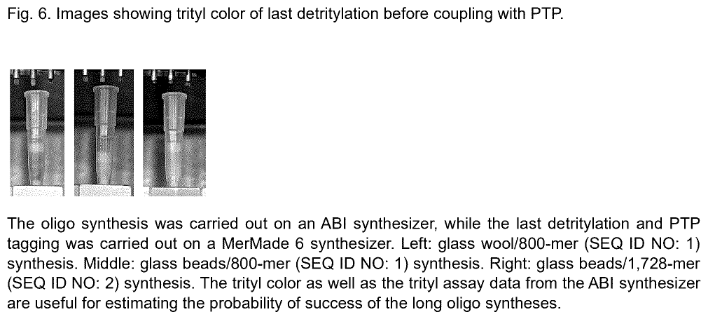

Because glass wool has about four times higher loading than glass beads, glass wool was chosen for our first trial for ODN synthesis. After testing for the synthesis of short ODNs, we used the glass wool support [F001] to synthesize the 800-mer GFP gene (SEQ ID NO: 001). The synthesis was conducted on an ABI 394 DNA/RNA synthesizer using 30 mg [F001] at a 29.4 nmol scale. It took about three days for the automated synthesis to complete. Trityl assays indicated that the average stepwise yield was above 99.6% and could reach 99.8%. To tag the full-length sequence with PTP for CBP purification, the synthesis was stopped at 799-mer. The trityl group of the 799-mer was removed by treating with the deblocking agent in a MerMade 6 synthesizer. Even after so many cycles, the orange trityl color could still be clearly observable (FIG. 6) [Yin et al 2025 Chem Sci 16:1966 doi:10.1039/d4sc06958g].

The last nucleotide at the 5′-end of the 800-mer (SEQ ID NO: 001) was introduced using PTP on the MerMade 6 synthesizer [Yin et al 2025 Chem Sci 16:1966 doi:10.1039/d4sc06958g], which also tagged the full-length sequence with the methacrylamide polymerizable group. For deprotection and cleavage, the glass wool was first treated with 10% DBU in ACN to remove the 2-cyanoethyl groups. This prevented potential side reactions between the side deprotection product acrylonitrile and deprotected ODN. Full deprotection and cleavage were then achieved under conventional conditions using concentrated NH₄OH. This gave the crude ODN product, a mixture of 5′-tagged full-length ODN and 5′-un-tagged failure sequences as well as other impurities (FIG. 1). CBP purification was then achieved as described earlier. The quantity of the ODN was determined to be 27.4 μg (111 pmol) for the synthesis involving 30 mg glass wool. The overall yield for the entire 800-mer synthesis and purification was 0.38%.

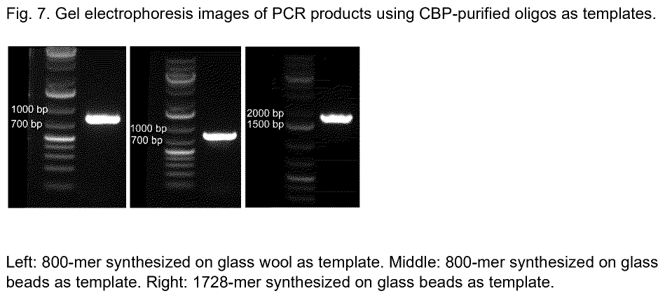

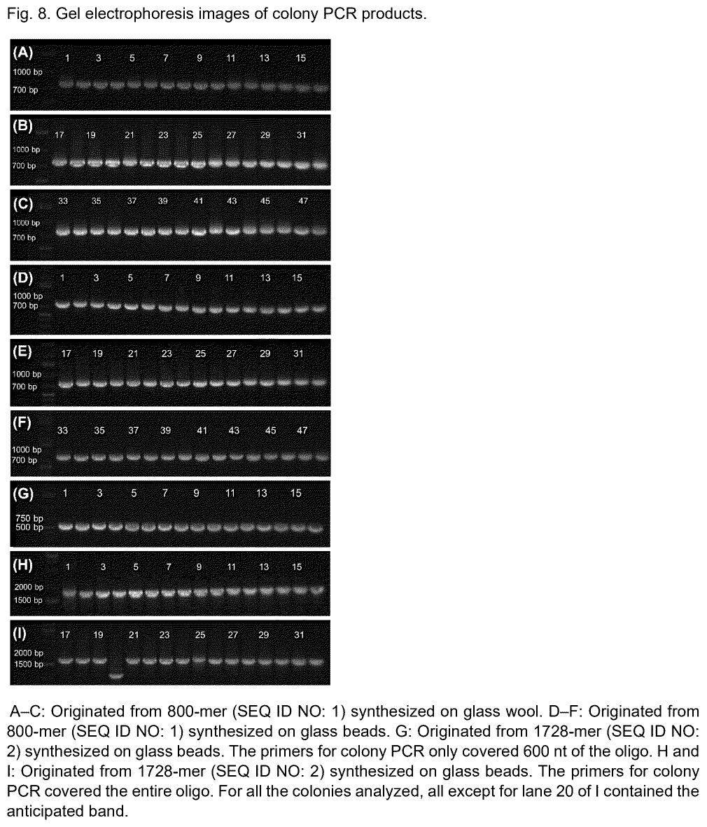

For characterization of the 800-mer (SEQ ID NO: 001), the same method involving PCR, gel electrophoresis, cloning, colony PCR, gel electrophoresis, and colony DNA Sanger sequencing described earlier was used (FIG. 2). More specifically, a portion (30 ng) of the CBP-purified 800-mer was subjected to PCR amplification using high fidelity DNA polymerase. The PCR product was analyzed with agarose gel electrophoresis. As shown in FIG. 7, a band corresponding to 800-mer can be clearly observed. A portion of the PCR product was ligated into the pCR™4Blunt-TOPO™ vector and transformed into Chemically Competent E. coli cells. The transformed cells were grown on agar plates. Colony PCR was performed on selected cell colonies. The PCR products were analyzed with agarose gel electrophoresis. As shown in FIG. 8A-C, all the 48 colonies contained an expected band. Plasmids of the colonies were subjected to Sanger sequencing. Among the 48 colonies sequenced, 41 contained the correct sequence, corresponding to a success rate of 85%. The errors in the incorrect sequences (SEQ ID NOs: 005-011) included three substitutions, four singe nucleotide deletions and one 10-nt deletion. The rates for the different errors were all lower than 0.003% except for single nucleotide deletion, which had a rate of 0.0104%. The sum of the error rates was 0.0208%, which is significantly lower than the value of 0.58% reported in the literature for 100-mer synthesis on CPG [Masaki et al 2022 Sci Rep 12:12095 doi:10.1038/s41598-022-16222-2].

Although the loading of glass beads is about four times lower than glass wool, the quantity of ODNs produced on them under typical small scale synthesis conditions is still predicted to be higher than 1 pmol, the quantity sufficient for most biological applications [Kuhn et al 2017 Eng Life Sci 17:6 doi:10.1002/elsc.201600121; Kim et al 2011 J Biotechno/151:319 doi:10.1016/j.jbiotec.2011.01.004]. For example, with 100 mg glass beads with a loading of 256 nmol/g, assuming an average stepwise yield of 99.7%, which corresponds to an overall yield of 0.25% for a 2,000-mer synthesis, the quantity of full-length oligo is ˜64 pmol. Considering that glass beads are easier to handle and less likely to generate fine particles that may block the lines of synthesizer, we decided to test long oligo synthesis on glass beads for the purpose of comparison with glass wool.

ODN synthesis was conducted under the same conditions as the case of glass wool. The same 800-mer (SEQ ID NO: 001) was used as the synthesis target. The scale was 12.8 nmol, for which 50 mg glass beads were used. Deprotection and cleavage as well as CBP purification were also the same except that only 20 mg (theoretically 5.12 nmol oligo) glass beads were used. The quantity of the ODN obtained was determined to be 168 ng (0.68 pmol) for the synthesis involving 20 mg glass beads. The overall yield for the entire 800-mer synthesis and purification was 0.013%, which is much lower than the value 0.38% obtained for glass wool. The reason is unclear but may be attributable to the loss of materials in the synthesis, deprotection, cleavage and purification process probably due to the increased difficulty in handling much smaller quantities of ODN.

The CBP purified 800-mer (SEQ ID NO: 001) was also characterized using the method involving PCR, gel electrophoresis, cloning, gel electrophoresis, and Sanger sequencing (FIG. 2). The image of the gel for electrophoresis analysis of the PCR product is shown in FIG. 7. Even though the quantity of oligos was much lower, the band corresponding to the 800-mer is clear. The image of the gel for analysis of colony PCR products is shown in FIG. 8D-F. As can be seen, all colonies selected for the analysis had the 800-mer sequence. Plasmids of 47 colonies were subjected to Sanger sequencing. Among the 47 colonies sequenced, 45 contained the correct sequence, which was 96%. The errors in the incorrect sequences only include one deletion (SEQ ID NO: 012) and one 2-nt deletion (SEQ ID NO: 013). The rates for both errors were 0.0027%. The sum of the error rates was 0.0054%, which was much lower than the value for glass wool (0.0208%) and that for CPG as reported in the literature for 100-mer synthesis (0.58%) [Masaki et al 2022 Sci Rep 12:12095 doi:10.1038/s41598-022-16222-2].

With the success of 800-mer synthesis, we decided to synthesize the 1,728-mer Φ29 DNA polymerase gene (SEQ ID NO: 002). Because glass beads had close to zero errors, and had a higher percentage of correct sequences than glass wool (96% vs. 85%) for the 800-mer syntheses, glass beads were chosen for the synthesis. The synthesis, deprotection, and CBP purification procedures as well as PCR, cloning, and sequencing were the same as described for glass wool. The scale of the synthesis was 33.28 nmol, which corresponds to 130 mg glass beads. The quantity of oligo obtained was 2.83 μg (5.28 pmol) for the synthesis using 130 mg glass beads (41 pmol/g). The overall yield for the entire 1,728-mer synthesis and purification was 0.016%, which is similar to that of 800-mer synthesis using glass beads as the support.

The image of the gel for electrophoresis analysis of the PCR product of the CBP purified 1,728-mer (SEQ ID NO: 002) is shown in FIG. 7. As can be seen, the expected band can be clearly observed. Using primers targeting only a portion of the 1,728-mer. All colonies were found to have the gene (FIG. 8G). Plasmids of the 16 colonies were subjected to Sanger sequencing. Among the 16 colonies sequenced, 7 contained the correct sequence, which corresponds to a success rate of 44%. The errors in the incorrect sequences (SEQ ID NOs: 014-022) include five substitutions, three single nucleotide deletion, two 2 nt deletion and 2 single nucleotide insertion. The sum of the error rates was 0.0434%. We also performed gel electrophoresis on colony PCR products of additional colonies using primers covering the entire 1,728-mer. Among 32 colonies, only one did not show the expected band (FIG. 8H and I).

With the aforementioned discoveries, it is obvious that conducting oligo synthesis on the solid supports comprising smooth surface as opposed to known methods, using which synthesis is conducted in the pores or in the matrix of soluble or swellable materials, can make a drastic difference. It can not only solve the steric hindrance problem, but also, unexpectedly, solve the formidable problem of high error rates for long oligo synthesis. Conducting oligo synthesis on smooth surface is non-obvious because it is against the common wisdom that oligo synthesis needs to be conducted in the pores of support to be practically useful in terms of product quantity. In addition, the finding that it drastically lowers the error rates was unexpected. For this finding, there is still not an explanation.

Therefore, provided are solid supports for oligo synthesis comprising substantially smooth surface characterized by the absence of intentional internal pores for the purpose of increasing surface area and oligonucleotide loading, wherein the solid support is physically configured to permit reagent flow through a packed bed of said solid support driven by gravity or pressure difference, or said solid support is the inner wall of an oligonucleotide synthesis column, tube or capillary, and wherein said smooth surface reduces steric hindrance during oligonucleotide synthesis and enables synthesis of oligonucleotides having a length of at least 100 nucleotides.

In some embodiments, the solid support comprises particles with shapes including but are not limited to sphere, fiber, rod, disc, square plate, cube, ellipsoid, cylinder, cone, pyramid, platelet, hexagonal prism, ribbon, toroid, sheet, thread, or clusters of particles with one or more of the shapes.

In some embodiments, the smooth surface is a portion of or the entire inner wall of an oligo synthesis column, tube, or capillary. Synthesis is conducted by running reagents through the column, tube, or capillary.

In some embodiments, the solid support is comprised of glass, carbon based material, or highly cross-linked polymer.

In some embodiments, the solid support is comprised of glass beads or glass wool.

In some embodiments, the solid support is comprised of carbon fiber.

In some embodiments, the solid support is comprised of highly cross-linked polystyrene.

In some embodiments, the solid support has a cleavable linker through which oligo is synthesized.

In some embodiments, the oligo to be synthesized comprises oligodeoxyribonucleotide (ODN), oligoribonucleotide (ORN), hybrid of ODN and ORN, modified oligo, or hybrid of unmodified and modified oligos. For the cases of ORN, hybrid of ODN and ORN, modified oligo, or hybrid of unmodified and modified oligos, the procedures are similar to the case of ODN. With the data presented for ODN, a person having ordinary skill in the art of oligo synthesis can readily adapt the new solid supports comprising smooth surface for their synthesis without much experimentation.

In some embodiments, oligo synthesis involves phosphoramidite chemistry.

In some embodiments, oligo synthesis involves enzymatic method or methods. The enzymatic methods include but are not limited to those reported in the literature [Hoose et al 2023 Nat Rev Chem 7:144 doi:10.1038/s41570-022-00456-9; Lee et al 2019 Nat Commun 10:2383 doi:10.1038/s41467-019-10258-1; Eisenstein 2020 Nat Biotechnol 38:1113 doi:10.1038/s41587-020-0695-9]. Replacing the solid supports used in enzymatic methods with the new solid support comprising smooth surface for oligo synthesis can be readily accomplished by a person having the ordinary skill in the art without much experimentation.

In some embodiments, oligo synthesis involves both chemical and enzymatic methods.

Also provided is a method of oligo synthesis, comprising steps (a) providing a solid support having a substantially smooth, non-porous surface; and (b) performing stepwise synthesis on the smooth surface to elongate the oligo chain, wherein said solid support is physically configured to permit reagent flow through a packed bed of said solid support in a column under gravity or pressure-driven flow, or said solid support is the inner wall of an oligonucleotide synthesis column, tube or capillary, and wherein the synthesis is conducted entirely on the smooth surface of said solid support, thereby reducing steric hindrance and enabling synthesis of oligos having a length of at least 100 nucleotides.

In some embodiments, the solid support of the oligo synthesis method comprises particles with shapes including sphere, fiber, rod, disc, square plate, cube, ellipsoid, cylinder, cone, pyramid, platelet, hexagonal prism, ribbon, toroid, sheet, thread, or clusters of particles with one or more of the shapes

In some embodiments, the smooth surface of the solid support of the oligo synthesis method is the inner wall of an oligo synthesis column, tube, or capillary.

In some embodiments, the smooth surface of the solid support of the oligo synthesis method comprises glass, fused silica, carbon based material, or highly cross-linked polymer.

In some embodiments, the solid support of the oligo synthesis method comprises a cleavable linker via which oligo is synthesized.

In some embodiments, the oligo synthesized using the method involving solid support with smooth surface is oligodeoxyribonucleotide (ODN), oligoribonucleotide (ORN), hybrid of ODN and ORN, modified oligo, or hybrid of unmodified and modified oligos. For the cases of ORN, hybrid of ODN and ORN, modified oligo, or hybrid of unmodified and modified oligos, the procedures are similar to the case of ODN. With the data presented for ODN, a person having ordinary skill in the art of oligo synthesis can readily adapt the new solid supports comprising smooth surface for their synthesis without much experimentation.

In some embodiments, the oligo synthesis method involves the use of the phosphoramidite chemistry.

In some embodiments, the oligo synthesis method involves the use of enzymatic reaction or reactions. The enzymatic methods include but are not limited to those reported in the literature [Hoose et al 2023 Nat Rev Chem 7:144 doi:10.1038/s41570-022-00456-9; Lee et al 2019 Nat Commun 10:2383 doi:10.1038/s41467-019-10258-1; Eisenstein 2020 Nat Biotechnol 38:1113 doi:10.1038/s41587-020-0695-9]. Replacing the solid supports used in enzymatic methods with the new solid support comprising smooth surface for oligo synthesis can be readily accomplished by a person having the ordinary skill in the art without much experimentation.

### EXAMPLE PROCEDURES

All the procedures in the Example Procedures section can be carried out by persons skilled in the art without excessive experimentations. The descriptions or examples provided herein are intended for illustrative purpose only and should not be construed as limiting the scope of the invention.

### Compound Synthesis

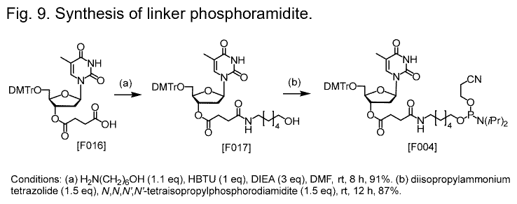

Synthesis of compound [F017] (FIG. 9). To a solution of compound [F016] [Wang et al 2021 Patent: CN101870717B] (2 g, 3.1 mmol, 1 eq) in dry DMF (20 mL) was added DIEA (1.2 g, 9.3 mmol, 3 eq) and HBTU (1.17 g, 3.1 mmol, 1 eq). The mixture was stirred under nitrogen at rt for 15 min. The content was transferred dropwise to flask containing the solution of 6-amino-1-hexanol (0.25 g, 3.41 mmol, 1.1 eq) in dry DMF (5 mL) via a cannula with its inlet terminus wrapped with cotton to prevent transfer of insoluble materials formed. After stirring at rt for 8 h, the mixture was concentrated to ˜30 mL under vacuum from an oil pump. The residue was purified by dissolving the sample in the solvent mixture of acetone/hexane 3:1 with 5% Et3N, loading onto a column (SiO₂), and eluting with the same solvent mixture. Compound [F017] was give as a pale yellow foam upon drying under high vacuum: 2.1 g, 91%; ¹H NMR (500 MHZ, CDCI₃) δ1.27-1.50 (m, 8H), 2.40-2.62 (m, 9H), 2.82 (s, 2H), 2.94 (s, 2H), 3.14-3.18 (m, 2H), 3.41 (s, 3H), 3.53-3.55 (t, J=5.0 Hz, 1H), 4.11 (s, 1H), 5.43 (s, 1H), 6.33-6.37 (m, 1H), 6.78-6.80 (d, J=10.0 Hz, 4H), 7.18-7.35 (m, 9H), 7.58 (s, 1H), 7.95 (s, 1H), 9.82 (s, 1H); ¹³C NMR (126 MHZ, CDCI₃) δ8.7, 25.2, 26.2, 29.1, 30.5, 31.3 32.2, 36.7, 47.2, 55.2, 62.4, 63.6, 75.4, 83.9, 84.3, 87.0, 111.4, 113.3, 127.1, 128.0, 130.0, 135.1, 135.7, 144.1, 150.6, 158.7, 163.1, 164.1, 171.4, 172.3.

Synthesis of compound [F004] (FIG. 9). To a solution of compound [F017] (2 g, 2.6 mmol, 1 eq) in dry ACN was added diisopropylammonium tetrazolide (0.69 g, 4.0 mmol, 1.5 eq) and 2-cyanoethyl N,N,N′, N′-tetraisopropylphosphorodiamidite (1.2 g, 4.0 mmol, 1.5 eq) at rt under nitrogen. After stirring overnight, the mixture was concentrated to dryness. The product was purified by dissolving in the solvent mixture of acetone/hexane 3:1 with 5% Et₃N, loading onto a column (SiO₂), and eluting with the same solvent mixture. The product [F004] was given as a white foam upon drying under high vacuum: 2.2 g, 87%; ¹H NMR (500 MHZ, CDCI3) δ1.10-1.12 (t, J=5.0 Hz, 12H), 1.18-1.54 (m, 8H), 2.40-2.68 (m, 8H), 3.12-3.16 (m, 1H), 3.40-4.08, (m, 14H), 5.43 (s, 1H), 6.25 (s, 1H), 6.32-6.39 (m, 1H), 6.76-6.78 (d, J=10.0 Hz, 4H), 7.14-7.34 (m, 9H), 7.55 (s, 1H); ¹³C NMR (126 MHZ, CDCI₃) δ11.5, 20.3, 22.8, 24.5, 25.5, 26.4, 29.3, 30.4, 31.0, 37.8, 39.4, 42.8, 45.2, 55.1, 58.2, 63.5, 75.4, 83.8, 84.2, 86.9, 111.4, 113.2, 117.8, 127.0, 128.0, 129.9, 135.1, 135.4, 144.1150.7, 158.6, 164.0, 171.0, 172.3; ³¹P NMR (202 MHZ, CDCI₃) δ146.97, 147.01.

### Procedure for the Functionalization of Glass Wool

Glass wool (Catalog Number G0034, Flinn Scientific, Inc., 2 g) was soaked in a piranha solution (H₂SO_{4/}H₂O₂ 3:1, v/v, ˜20 mL) with occasional shaking at rt [Khanal et al 2016 Chem Eur J 22:9760 doi:10.1002/chem.201600982]. Caution, piranha solution is highly reactive and corrosive; appropriate safety procedure must be followed. After 1 h, the supernatant was removed. The glass wool was washed sequentially with water and ACN each for 5 times, and then allowed to dry in the air.

The activated glass wool [F005] (2 g, FIG. 3) in the solution of 1% 2-[(acetoxy(polyethyleneoxy)propyl]triethoxysilane [F002] in freshly distilled toluene (20 mL) was incubated at rt for 20 min. The supernatant was removed, and the glass wool was washed with toluene 5 times and then incubated in an oven at 100° C. for 4 h. After cooling to rt, the glass wool was washed with chloroform to give the functionalized glass wool [F006] (FIG. 3).

To remove the acetyl group, the glass wool [F006] (1 g) was incubated in NH₄OH (30%, 10 mL) at 55° C. for 2 h. The supernatant was removed, and the glass wool was washed sequentially with water and ACN each for 5 times, and then allowed to dry in the air. This gave [F007] (FIG. 3).

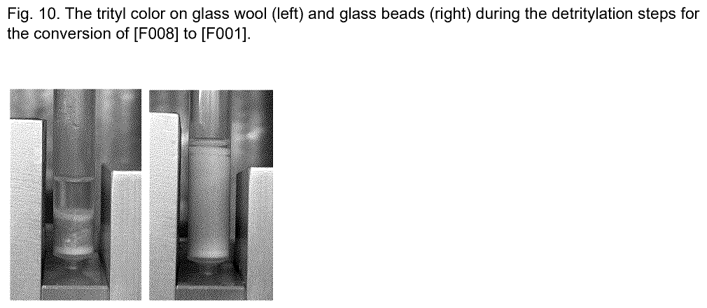

The glass wool [F007] (1 g) was packed in a DNA synthesis column. On a MerMade 6 synthesizer, the following steps were carried out. Between the steps, the glass wool was washed with dry ACN. Coupling: [F003] (0.5 mL, 0.1 M in ACN), DCI (0.5 mL, 0.25 M in ACN), 10 min waiting; repeat 4 times. Oxidation: I₂ (1 mL, 0.02 M in THF/pyridine/H₂O), 1 min waiting; repeat 2times. Deblocking: DCA (1 mL, 2% in DCM), 90 sec waiting; repeat 3 times. The coupling and oxidation steps were repeated 1 time. This converted [F007] to [F008]. The deblocking (see FIG. 10 for color), coupling (using instead of [F003]), and oxidation steps were repeated. This converted [F008] to [F001]. The loading was determined to be 981 nmol/g using a reported method involving treating the glass wool with an acid and measuring the UV absorption of trityl cations [Guzaev et al 2013 Curr Protoc Nucleic Acid Chem doi:10.1002/0471142700.nc0302s52].

### Procedure for the Functionalization of Glass Beads

Glass beads (5 g) were functionalized using the same conditions as described for glass wool functionalization. The loading was determined to be 256 nmol/g. The functionalized glass beads are also represented by [F001].

### Procedure for the Functionalization of Carbon Fiber

Carbon fiber (Product Number, PX30MF0150, Milled Fiber, 150 μm length, 7.2 μm diameter, Zoltek, Toray Group, Bridgeton, MO; 10 g) was treated with acetone under reflux conditions under nitrogen for 12 h (FIG. 4). After cooling to rt, the supernatant was removed. The carbon fiber was washed sequentially with acetone, chloroform, acetone, methanol and water. In a round-bottomed flask containing the acetone-treated carbon fiber, concentrated HNO₃ (50 mL) was added, and mixture was heated at 120° C. for 12 h under nitrogen. No stirring was applied. After cooling to rt, the supernatant was removed. The carbon fiber [F009] was washed sequentially with water, acetone, chloroform, acetone, and chloroform. Thionyl chloride (30 mL) was added, and mixture was heated at reflux temperature for 5 h under nitrogen. No stirring was applied. Under nitrogen atmosphere, the carbon fiber was washed sequentially with dry chloroform and THF. This gave [F010]. To [F010] under nitrogen was added the solution of [F011] (2.0 g) in THF. The mixture was heated at reflux temperature for 12 h. No stirring was applied. After cooling to rt, the supernatant was removed, and the product [F012] was washed with THF, chloroform, and THF (FIG. 4).

To carbon fiber [F012] (FIG. 4) suspended in THF (30 mL) in a pressure tube was added NaH (60% in mineral oil, 27 mg) and ethylene oxide solution in THF (2.5-3.3 M, 1 mL). The mixture was stirred at rt for 1 h and then heated in a 110° C. oil bath for 5 h. After cooling to rt, the supernatant was removed. The carbon fiber was washed with THF, acetone, chloroform, and ACN sequentially. This converted [F012] to [F013].

[F013] was converted to [F014] on a MerMade DNA/RNA synthesizer under typical DNA synthesis conditions using phosphoramidite monomers [F003] and [F004] with the following exceptions (FIG. 4). In the synthetic cycles involving [F003], no capping was conducted. In all synthetic cycles, the coupling time was seven times longer than standard conditions. [F014] was washed with ACN on the synthesizer extensively, and dried by applying vacuum provided by the synthesizer. It is noted that when the carbon fiber is dry, mechanical force should be avoided as much as possible because dry carbon fiber may be fragile.

### Procedure for 800-mer Oligo (SEQ ID NO: 001) Synthesis on Glass Wool

The glass wool [F001] (30 mg, 29.4 nmol) was packed in an empty 0.2 μmol synthesis column, and loaded onto an ABI 394 DNA/RNA synthesizer. The synthesizer manufacturer recommended 1 μmol synthetic cycle with slight modifications was used. The specific conditions were the following. Deblocking: DCA (2% in DCM), 98 sec. Coupling: Bz-dA, Ac-dC, iBu-dG or dT phosphoramidite (0.1 M in ACN); DCI (0.25 M in ACN), 2.5 sec×2 reagent delivery, 35 sec waiting. Capping: Cap A, THF/pyridine/AC₂O; cap B, 1-methylimidazole (16% in THF), 10 sec delivery, 10 sec waiting. Oxidation: I₂ (0.02 M in THF/pyridine/H₂O), 8 sec delivery, 15 sec waiting. Washing conditions between the steps were the same as recommended by the synthesizer manufacturer except for an additional wash with ACN after the oxidation step to ensure complete washing. For purposes such as refilling reagents, the synthesis was set up as several shorter ones such as 200-mer. The average stepwise yields as indicated by the trityl assay of the synthesizer were consistently 99.6%, 99.7% or 99.8% after the syntheses reached over 100 synthetic cycles. The last nucleotide was incorporated with the polymerizable tagging phosphoramidite PTP (FIG. 1), of which preparation procedure could be found in the literature [Pokharel et al 2016 Green Chem 18:1125 doi:10.1039/c5gc01762a; Fang et al 2024 Curr Protoc 4: e70028 doi:10.1002/cpz1.70028; Yin et al 2024 PeerJ Org Chem 6:e12 doi:10.7717/peerj-ochem.12], where B is thymine, on a MerMade 6 synthesizer (0.1 M in ACN, 0.25 M DCI in ACN, 5 min; repeat 3 times; followed by capping and oxidation as usual; no detritylation). The detritylation of the 799th nucleotide before the coupling step involving PTP was also performed on the MerMade 6 synthesizer. The trityl color was visible (FIG. 6).

### Procedure for the Deprotection and Cleavage of 800-mer Oligo (SEQ ID NO: 001) Synthesized on Glass Wool

To the glass wool (˜30 mg, theoretically 29.4 nmol oligo) in a 1.5 mL centrifuge tube was added DBU (10% in ACN). The tube was gently shaken at rt for 10 min. The supernatant was removed, and the DBU treatment was repeated 1 time. After the supernatant was removed, the glass wool was washed with ACN for 5 times. After washing with water, saturated NH₄OH (0.5 mL) was added. The tube was sealed and heated at 55° C. for 16 h. After cooling to rt, the supernatant was transferred to a clean centrifuge tube. The glass wool was washed with water (200 μL×3). The supernatant and washes were combined, and the volume was adjusted to 50 μL by evaporation and dilution.

### Procedure for CBP Purification of 800-mer Oligo (SEQ ID NO: 001) Synthesized on Glass Wool

The procedure involves Polymerization, Washing, and Cleavage [Pokharel et al 2016 Green Chem 18:1125 doi:10.1039/c5gc01762a; Fang et al 2010 Org Lett 12:3720 doi:10.1021/ol101316g; Yuan et al 2012 RSC Adv 2:2803 doi:10.1039/c2ra01357f; Eriyagama et al 2018 Org Process Res Dev 22:1282 doi:10.1021/acs.oprd.8b00209]. Polymerization: To the 50 μL solution of crude oligo containing tagged full-length oligo and failure oligos (FIG. 1) as well as other impurities (theoretically 29.4 nmol oligo) in a 1.5 mL centrifuge tube was added 12 μL polymerization solution (N,N-dimethylacrylamide, 340.5 L, 3.32 mmol; N,N′-methylenebis(acrylamide), 17 mg, 0.11 mmol; sodium acrylate, 3 mg, 0.032 mmol; water, 170.5 μL). After mixing, ammonium persulfate (0.23 M, 5 μL) and N,N,N′, N′-tetramethylethylenediamine (TMEDA, 0.69 M, 5 μL) were added. The tube was closed, and the content was quickly mixed with a short vortex and spin. The polymerization reaction was allowed to proceed at rt for 1 h. Washing: The gel (˜100 μL in size) was transferred into a 50 mL centrifuge tube. NaOAc solution (20%, pH unadjusted, 20 mL) was added. The mixture was gently shaken at rt overnight. The gel was taken to another 50 mL centrifuge tube, and Et3N solution (5%, 20 mL) was added. The tube was shaken at rt overnight. The gel was taken out, placed into a 2 mL centrifugal filter unit over the filter, and cut into small pieces using a spatula. The gel was washed with water (0.5 mL×5). Cleavage: To half volume of the gel, minimum AcOH (80%, ˜200 μL) that could cover the gel was added. The mixture was incubated at rt for 5 min with occasional shaking. The liquid and gel were separated by centrifugation. The liquid was diluted with water (800 μL) to minimize the possibility of oligo damage by acid. The treatment of the gel with acid was repeated two times. The gel was washed with water (0.3 mL×3). The bottom of the filtering unit holding the gel was stopped with parafilm, and to the gel was added water (500 μL). The mixture was shaken at rt overnight. The solution and gel was separated by centrifugation, and the gel was washed with water (250 μL×2). These gave the oligo solutions in five tubes (˜1 mL each). The solutions were concentrated, combined and evaporated to dryness. The residue acid is suggested to be removed by precipitation of the oligo from NH₄OH solution by nBuOH [Fang et al 2011 Chem Commun 47:1345 doi:10.1039/c0cc04374e] if the oligo needs to be stored for more than two days. The oligo (theoretically 14.7 nmol) was dissolved in water (10 uL). Quantification using Qubit 4 Fluorometer indicated that 13.7 μg (55.56 pmol) oligo was obtained. The overall yield for the 800-mer synthesis and purification was 0.38%.

### Procedure for PCR Amplification of 800-mer Oligo (SEQ ID NO: 001) Synthesized on Glass Wool

The mixture of CBP-purified oligo (˜30 ng as quantified with Qubit 4 Fluorometer), Thermo Scientific™ Phusion™ High-Fidelity DNA Polymerase (0.4 U), forward and reverse primers (SEQ ID NOs: 023 and 024, 0.5 μM each), HF buffer (1×), dNTPs (0.2 mM each), and nuclease-free water (20 μL total mixture volume, concentrations were final) was subjected to the following PCR cycles: 98° C. for 30 sec for initial denaturing; 98° C. for 7 sec, 58° C. for 15 sec, and 72° C. for 25 sec for 32 cycles; and 72° C. for 7 min. The PCR product was analyzed with agarose gel (1%) electrophoresis (GelRed® staining). Gel image is given in FIG. 7.

### Procedure for Cloning and Sanger Sequencing the 800-mer Oligo (SEQ ID NO: 001) Synthesized on Glass Wool

The PCR product (3 μL out of 20 μL) was ligated into the pCR™4Blunt-TOPO™ vector following manufacturer's protocol. The resulting recombinant DNA was then transformed into One Shot Mach1 T1 Phage-Resistant Chemically Competent E. coli following manufacturer's protocol. The transformed cells (50 μL) were spread over kanamycin containing agar plates, which was incubated in a 37° C. incubator overnight. A portion of selected colonies was harvested using a sterile pipette tip and transferred into 50 μL of lysis buffer (1% triton X-100, 20 mM Tris, pH 8.0, 2mM EDTA, pH 8.0). The lysis solutions were then heated at 95° C. for 10 min. Colony PCR reactions were set up using the recipe: DreamTaq PCR Mater Mix (1x), 2 uL colony lysis solution, primers SEQ ID NOs: 023 and 024, (0.5 μM each), and water to 20 μL (all concentrations are final). The samples were then subjected to the following PCR conditions: 95° C. for 2 min for initial denaturing; 95° C. for 30 sec for denaturing, 57° C. for 30 sec for annealing, and 72° C. for extension for 1 min for 32 cycles; 72° C. for extension for 7 min. The PCR products were analyzed with electrophoresis on a 1% agarose gel (FIG. 8A-C). Plasmid DNA from additional portions of the colonies that showed expected band in the colony PCR analysis were sent to MCLAB for Sanger sequencing. Both forward (SEQ ID NO: 025) and reverse (SEQ ID NO: 026) reads were obtained, which were assembled into a single contig using the Cap3 program (https://doua.prabi.fr/software/cap3). Several colonies that were unable to generate contigs due to poor sequencing quality were not included here. The contigs that were generated successfully were aligned to the reference sequence using the BLAST alignment software to trim off vector sequence and were then aligned with the reference sequence using CLUSTAL Omega. The data are in the XML file. See SEQ ID NOs: 001 and 005-011.

### Procedure for the Synthesis, Cleavage and Deprotection, PCR, Cloning and Sanger Sequencing of the 800-mer GFP Gene (SEQ ID NO: 001) and the 1,728-mer Φ29 DNA Polymerase Gene (SEQ ID NO: 002) Synthesis on Glass Beads

Using glass beads as the solid support, the 800-mer GFP gene (50 mg glass beads, theoretically 12.8 nmol) and the 1,728-mer Φ29 DNA polymerase gene (130 mg glass beads, 33.28 nmol) were synthesized under the same conditions described above for the synthesis of the 800-mer GFP gene on glass wool.

The 800-mer GFP (SEQ ID NO: 001) and 1,728-mer ¢29 DNA polymerase (SEQ ID NO: 002) genes synthesized on glass beads were purified using the same CBP procedure described above for the purification of the 800-mer GFP gene synthesized on glass wool. For the 800-mer, all the crude oligo (theoretically 5.12 nmol) was subjected to the polymerization for CBP. Half of the polyacrylamide gel was subjected for cleavage. Quantification of the purified oligo (theoretically 2.56 nmol) with Qubit 4 Fluorometer indicated that 84 ng (0.34 pmol) oligo was obtained. The overall yield for the 800-mer synthesis and purification was 0.013%. For the 1,728-mer, all the crude oligo (theoretically 33.28 nmol) was subjected to the polymerization. Half of the gel was subjected to cleavage. Quantification of the purified oligo (theoretically 16.64 nmol) with Qubit 4Fluorometer indicated that 1.416 μg (2.64 pmol) oligo was obtained. The overall yield for the 1,728-mer synthesis and purification was 0.016%.

The same PCR conditions described above for the PCR amplification of the 800-mer GFP (SEQ ID NO: 001) gene synthesized on glass wool were applied to the 800-mer GFP gene synthesized on glass beads. For the 1,728-mer Φ29 DNA polymerase gene, the PCR was conducted similarly except for the following modifications: primers were SEQ ID NOs: 027 and 028; template quantity was ˜7 ng; and the PCR cycles were modified so that the annealing step was 60.6° C. for 10 seconds and the following elongation step was 72° C. for 30 sec. Gel images of the PCR products are given in FIG. 7.

The cloning and sequencing procedures for the 800-mer GFP gene synthesized on glass beads were exactly the same as described for the 800-mer gene synthesized on glass wool. Gel images of colony PCR products are given in FIG. 8D-F. For the 1,728-mer Φ29 DNA polymerase gene synthesized on glass beads, the following adjustments were applied. For colony PCR, the primers SEQ ID NOs: 029 and 030, which covered a 600-mer region within the 1,728-mer gene, were used with an annealing temperature of 57.5° C. Gel image of PCR products is given in FIG. 8G. For Sanger sequencing and data analysis, four sequencing reads were generated for each plasmid, which was from the colonies giving bands in FIG. 8G, using primers SEQ ID NOs: 029 and 030 and SEQ ID NOs: 025 and 026. All four reads were used to generate the contig. Among the 16 sequenced colonies, seven had the correct sequence SEQ ID NO: 002. Nine had one or more errors SEQ ID NOs: 014-022. Colony PCR was also carried out using primers SEQ ID NOs: 027 and 028, which covered the entire 1,728-mer gene. The annealing temperature was 55.3° C. The gel images of the PCR products are given in FIG. 8H-I.

### Procedure for the Synthesis of 100-mer (SEQ ID NO: 031) on Carbon Fiber

The 100-mer (SEQ ID NO: 031), which is a portion of the eGFP gene [Zhang et al 2023 iScience 26:107739 doi:10.1016/j.isci.2023.107739] was synthesized on carbon fiber support [F014] (FIG. 4). The conditions were the same as those described for 800-mer GFP gene (SEQ ID NO: 001) synthesis on glass wool except that for the incorporation of the first ten nucleotides, the coupling time was seven times longer. Trityl assay indicated excellent coupling yields. RP HPLC and MALDI MS analysis proved the synthesis was successful. We also tested the version of the carbon fiber support [F014] with n equals 3, which was produced using the procedure in FIG. 4 without the step from [F012] to [F013], for the 100-mer ODN (SEQ ID NO: 031) synthesis. No ODN product was found by HPLC and MALDI MS analyses.

1. A solid support for the synthesis of oligonucleotides, comprising:
a substantially smooth surface characterized by the absence of intentional pores for the purpose of increasing surface area and oligonucleotide loading,
wherein said solid support is physically configured to permit reagent flow through a packed bed of said solid support driven by gravity or pressure difference, or said solid support is the inner wall of an oligonucleotide synthesis column, tube or capillary, and
wherein said smooth surface reduces steric hindrance during oligonucleotide synthesis and enables synthesis of oligonucleotides having a length of at least 100 nucleotides.

2. The solid support of claim 1 wherein said oligonucleotides have at least 200 nucleotides.

3. The solid support of claim 1 wherein said oligonucleotides have at least 400 nucleotides.

4. The solid support of claim 1 that comprises particles with shapes selected from the group consisting of sphere, fiber, rod, disc, square plate, cube, ellipsoid, cylinder, cone, pyramid, platelet, hexagonal prism, ribbon, toroid, sheet, thread, or clusters of particles with one or more of the shapes.

5. The solid support of claim 1 wherein said smooth surface is the inner wall of an oligonucleotide synthesis column, tube, or capillary.

6. The solid support of claim 1 wherein said support comprises glass, carbon based material, or highly cross-linked polymer.

7. The solid support of claim 1 wherein said support comprises glass beads or glass wool.

8. The solid support of claim 1 wherein said support comprises carbon fiber.

9. The solid support of claim 1 wherein said support comprises highly cross-linked polystyrene.

10. The solid support of claim 1 having a cleavable linker on which oligonucleotide is synthesized.

11. The solid support of claim 1 wherein said oligonucleotide comprises oligodeoxyribonucleotide, oligoribonucleotide, hybrid of oligodeoxyribonucleotides and oligoribonucleotide, modified oligonucleotide, or hybrid of unmodified and modified oligonucleotide.

12. The solid support of claim 1 wherein said oligonucleotide is synthesized using a method or methods comprising the phosphoramidite chemistry.

13. The solid support of claim 1 wherein said oligonucleotide is synthesized using a method or methods comprising enzymatic method.

14. The solid support of claim 1 wherein oligo synthesis involves using both chemical and enzymatic methods.

15. A method for synthesizing oligonucleotides, comprising:
(a) providing a solid support having a substantially smooth, non-porous surface; and
(b) performing stepwise synthesis on said smooth surface to elongate the oligonucleotide chain,
wherein said solid support is physically configured to permit reagent flow through a packed bed of said solid support in a column under gravity or pressure-driven flow, or said solid support is the inner wall of an oligonucleotide synthesis column, tube or capillary, and
wherein the synthesis is conducted on the smooth surface of said solid support, thereby reducing steric hindrance and enabling synthesis of oligonucleotides having a length of at least 100 nucleotides.

16. The method of claim 14 wherein said length of oligonucleotide is at least 200 nucleotides.

17. The method of claim 14 wherein said length of oligonucleotide is at least 400 nucleotides.

18. The method of claim 14 wherein said solid support comprises particles with shapes selected from the group consisting of sphere, fiber, rod, disc, square plate, cube, ellipsoid, cylinder, cone, pyramid, platelet, hexagonal prism, ribbon, toroid, sheet, thread, or clusters of particles with one or more of the shapes.

19. The method of claim 14 wherein said smooth surface is the inner wall of an oligonucleotide synthesis column, tube, or capillary.

20. The method of claim 14 wherein said smooth surface comprises glass, fused silica, carbon based material, or highly cross-linked polymer.

21. The method of claim 14 wherein said solid support comprises a cleavable linker via which oligonucleotide is synthesized.

22. The method of claim 14 wherein said oligonucleotide is oligodeoxyribonucleotide, oligoribonucleotide, hybrid of oligodeoxyribonucleotide and oligoribonucleotide, modified oligonucleotide, or hybrid of unmodified and modified oligonucleotides.

23. The method of claim 14 wherein the phosphoramidite chemistry is involved in the synthesis.

24. The method of claim 14 wherein one of more enzymatic reactions are involved in the synthesis.
